(19) United States
(12) Patent Application Publication
(10) Pub. No.: US 2016/0081763 A1
(43) Pub. Date: Mar. 24, 2016

(54) PATHOLOGY LOCALIZER AND THERAPEUTICAL PROCEDURE GUIDE SYSTEM

(71) Applicants: Georgios AREALIS, Chalandri Attikis (GR); Eleftherios KOSMIDIS, Dafni Attikis (GR)

(72) Inventors: Georgios AREALIS, Chalandri Attikis (GR); Eleftherios KOSMIDIS, Dafni Attikis (GR)

(21) Appl. No.: 14/787,145

(22) PCT Filed: Apr. 26, 2013
(86) PCT No.: PCT/GR2013/000023

§ 371 Date: Oct. 26, 2015

(51) Int. Cl. A61B 19/00; A61B 6/00; A61B 6/03; A61B 5/055; A61B 6/12

(52) U.S. Cl. CPC A61B 19/54; A61B 19/50; A61B 5/055; A61B 6/12; A61B 6/032; A61B 6/5211; A61B 2019/5404; A61B 2019/5466; A61B 2019/5454; A61B 2019/507; A61B 2019/5491

(57) ABSTRACT

A pathology localizer and therapeutical procedure guide system comprising an elongate strip (1) provided with an array of three linear columnar members (2,3,4) having a common point of origin (O) at one end of strip (1) and extending divergently to corresponding ends (X,Y,Z) at the other end thereof. These linear columnar members (2,3,4) are equipped with a contrast medium visible in imaging modalities of a body portion with strip (1) placed thereupon, thereby producing an array of visible imprints of three linear segments (OX), (OZ), (OY) in a frontal X-Ray image and a series of visible imprints of three sequential spots (X₁,Z₁,Y₁) in a sectional CT or MRI image, wherein these visible imprints are used in precisely determining the locus of a pathology, if any, identified within the body portion being scanned and in subsequently determining an accurately defined path of instrumentation adapted to effect a therapeutical procedure therein.

### THE FIELD OF THE ART

The present invention is directed to a pathology localizer and accordingly a therapeutical procedure guide system for determining a penetration route of an invasive instrument or a radiation route of radiotherapy instrumentation to perform an intervention at a targeted area without adverse effects of either repetitive punctures or excessive radiation having damaging effects on living tissues and organs.

### THE BACKGROUND OF THE INVENTION

In conventional diagnosis of pathologies in the body of humans or animals, appropriate sectional images are obtained using computer tomography (CT) or magnetic resonance imaging (MRI), etc., such sectional images being further employed when deemed appropriate by a doctor to determine the most suitable entry point and the puncture path for leading an invasive instrument at the area subject to treatment, whilst the puncture probe of the invasive instrument is freehandedly directed into the body on the basis of a visual approximate estimate of the necessary entry angle. Thereafter repeated further images are obtained to verify appropriate penetration angle and path of the invasive instrument and puncture is being repeated until an acceptable entry path is being achieved. This conventionally necessary repetition of scanning the body using CT or MRI techniques leads to a variety of drawbacks, among them the damage caused by the repeated radioactive radiation to both the patient and the nursing personnel and the substantially increased time and cost necessary to complete the intervention. These drawbacks and disadvantages of conventional targeted surgery have led to evolution of stereotactic devices, capable of providing accurate locating of a guide path for the invasive instrument.

Stereotaxis relates to the accurate locating of a point in space, the term being used to define a surgical technique, also called stereotactic surgery, that uses medical imaging to precisely locate in three dimensions an anatomical site to which an invasive instrument or a beam of radiation is directed. Stereotactic surgery is a minimally invasive form of surgical intervention which makes use of a three-dimensional coordinate system to locate small targets inside the body and to perform on them a desired action, such as ablation, biopsy, lesion, injection, stimulation, implantation, radiosurgery (SRS), etc. Plain X-ray images, computed tomography, and magnetic resonance imaging can be used to guide the procedure. Stereotactic frame systems have been developed in relation to brain surgery in particular, wherein a mechanical device having head-holding clamps and bars which put the head in a fixed position in relation to a three-dimensional coordinate system (the so-called zero or origin). In small laboratory animals, these are usually bone landmarks which are known to bear a constant spatial relation to soft tissue. In humans, the reference points, as described above, are intracerebral structures which are clearly discernible in a radiograph or tomogram. The device is further provided with guide bars in the x, y and z directions, fitted with high precision vernier scales, which allow the neurosurgeon to position the point of a probe (an electrode, a cannula, etc.) inside the brain, at the calculated coordinates for the desired structure, through a small hole in the skull. Whilst in theory, any organ system inside the body can be subjected to stereotactic surgery, difficulties in setting up a reliable frame of reference (such as bone landmarks which bear a constant spatial relation to soft tissues) mean that its applications have been limited to brain surgery. Furthermore, the awkward feeling of the aforementioned mechanical device mounted onto the head of the patient has led to research in the direction of frameless stereotaxy.

Thus, U.S. Pat. No. 5,662,111 discloses a process of stereotactic optical navigation, providing a simultaneous display of representations showing the positional relationship of an invasive instrument in a computer graphic display of a patient's anatomy, wherein is being employed a system for quantitative computer graphic determination of positions on a patient's anatomy and positions on associated equipment located near the patient's anatomy in relation to anatomical data as from CT or MR scanning In particular the system proposed comprises a plurality of cameras for optically scanning plural fields of view containing the anatomy of the patient and of the invasive instrument, thereby providing location data on the invasive instrument related to the patient's anatomy and referenced in camera coordinates and subsequently combining transformed invasive instrument data and stereotactic image scanner data to form combined display data referenced in the scanner data coordinates to provide display signals and to drive a computer graphics display with these combined display data to simultaneously display an image representative of the invasive instrument related to the patient's anatomy.

US-2007/0036274 discloses a computed tomography unit having a marking device for the accurate marking of an intervention position by means of a laser beam that is being emitted onto an object under examination and a rotary frame holding a recording system, wherein the marking device is being assigned to the recording plane of the recording system, whereby specification of the positions of the marking device and of the positions identified by an operator is made possible for a surgical procedure to be likewise specified in the same coordinate system in an image being produced during a planning scan and subsequently it is made possible to calculate and adjust positions of the marking device with a scope of marking the intervention path into the object under examination.

U.S. Pat. No. 8,182,149 further discloses a method and marking out apparatus for marking out a guide line for a penetration instrument entering an object, comprising determining the penetration channel into the object based on image data generated by an imaging recording, e.g. a CT system, the penetration channel being defined by a penetration point into the object and at least one penetration direction, and emitting at least two light fan beams, preferably laser fanned beams, from different directions so that the line of intersection of the fan beams is coaxial with the penetration direction, wherein measurement data for determining the position of the penetration instrument is acquired by the imaging recording system and the alignment of the fan beams is accordingly corrected depending on a position of the penetration instrument.

The methods and devices proposed in the prior art as cited hereinabove make use of complex and accordingly expensive mechanisms to enable a guided penetration of an invasive instrument into a specific region of the body with a scope of reaching at an intervention site without endangering undesirable damaging of adjacent matter. Accordingly such methods and devices require a substantial investment for the purchase and installation thereof and an extensive training of personnel assigned to operate the same. It is therefore expected that their use will in practice be restricted in only a few highly equipped hospital units and that it cannot become the state of the art for an all-encompassing stereotactic approach of guided introduction of an invasive instrument into an object at the vicinity of a specific site wherein a planned intervention is being performed. Notwithstanding their extensive cost in hardware and training, some of the cited devices of the prior art still necessitate an undesirable repetition of CT or other imaging recording shots.

### SUMMARY OF THE INVENTION

It is the object of the present invention to propose a pathology localizer and accordingly a guide system for proceeding with treatment of the pathology by means of surgery, radiotherapy or else, said pathology localizer and treatment guide being a low cost device that is handy to use in an unlimited variety of circumstances wherein a diagnostic localization of a pathology in the body of humans or animals is required and henceforth in a broad variety of necessary surgical or other therapeutical procedures with a scope of accurately determining an appropriate guide path for the insertion of an invasive instrument or other therapeutical means at a targeted site for the performance of a therapeutical procedure, wherein such guide system does not necessitate any investment whatsoever in equipment and in training and can be practiced by all doctors and provide equitably satisfactory results.

According to one embodiment of the invention, the pathology localizer and therapeutical procedure guide system comprises a generally rectangular elongate strip being provided with an arrangement of three linear columnar members with a common point of origin at one end of the strip that diverge as they extend in the elongate direction till the other end of the same, said guide strip being attached onto a selected region of the body that is being subjected to CT or MRI or other imaging examination with an orientation such that each sectional image obtained in the course of this imaging examination will incorporate visual representations of intersecting said three linear columnar members extending in the elongate direction of the strip that will be depicted in the form of three spots on each sectional image being taken, such three spots lying along a straight line and defining a pair of line segments that is uniquely associated with a corresponding pair of line segments onto said guide strip.

According to the embodiment described hereinabove, the method of accurately determining an appropriate guide path for the insertion of an invasive or radiotherapy instrument at a targeted site for the performance of a therapeutical procedure of the present invention comprises:

A first step in which the doctor examines the patient and determines the appropriate CT or MRI or other type of scanning required, e.g. in the thoracic or in the lumbar spine with a scope of identifying a suspected pathology.

A second step in which an appropriately sized guide strip is selected and attached along the area that will be subjected to the prescribed CT or MRI or other type of scanning.

A third step in which the prescribed CT or MRI or other type of scanning is being performed and a plurality of sectional images are obtained, each image including a series of three sequential spots that constitute the visual representations of the cross-sectionally directed beam emitted by the CT or MRI scanning apparatus intersecting said three linear columnar members extending in the elongate direction of the guide strip.

A fourth step of evaluation of the plurality of sectional images being obtained to select the most appropriate sectional image wherein is located the specific area wherein the suspected pathology is being identified and wherein a subsequent therapeutical procedure will be required.

A fifth step of measurement of the pair of consecutive line segments determined by the three sequential spots that constitute the visual representations of the cross-sectionally directed beam emitted by the CT or MRI scanning apparatus intersecting said three linear columnar members extending in the elongate direction of the guide strip in this particularly selected sectional image.

A sixth step of locating the pair of linear segments across the array of three columnar members extending in the elongate direction of the guide strip that accurately corresponds to the measurement of the consecutive line segments determined by the three sequential spots in the sectional image that has been selected hereinabove, thereby establishing a specific linear locus onto the area of the body subjected to imaging, such linear locus accurately defining the coordinates of the identified pathology.

A seventh step during which, if a therapeutical subsequent surgical or radiotherapy procedure is deemed necessary, a guide path for the therapeutical instrumentation being used in the procedure to reach the specified area of the identified pathology is accurately determined following selection of an entrance point along the hereinabove specific linear locus and measuring the angle and depth of penetration through measurement of the distance of the selected entrance point from the targeted point of the identified pathology as depicted in the sectional image that has been selected as most appropriately depicting the identified pathology.

### BRIEF DESCRIPTION OF THE DRAWINGS

The invention will be best understood by those skilled in the art by reference to the accompanying drawings in which:

FIG. 1 shows a guide strip used as a pathology localizer and therapeutical procedure guide system in accordance with one preferred embodiment of the invention.

FIG. 2 shows a guide strip used as a pathology localizer and therapeutical procedure guide system in accordance with an alternative preferred embodiment of the invention.

FIG. 3 shows a perspective view of one illustrative preferred embodiment of an assembled guide strip product of FIG. 1 and a sectional view of the constituent parts thereof.

FIG. 4 shows a perspective view of another illustrative preferred embodiment of an assembled guide strip product of FIG. 1 and a sectional view of the constituent parts thereof.

FIG. 5 shows the guide strip being applied along a specified area of the spine of a human body.

FIG. 6 shows a selected sectional image taken by appropriate scanning equipment most appropriately depicting a pathology being identified along the spine, in a specific vertebra, such sectional image incorporating the consecutive three spots that define the specific linear locus of the identified pathology.

### DESCRIPTION OF PREFERRED EMBODIMENTS OF THE INVENTION

The invention will be hereinafter described by reference to the illustrative embodiments presented in the accompanying drawings.

A guide strip is proposed to serve as a pathology localizer employed in combination with various conventional diagnostic imaging of the body (CT, MRI, X-RAY, angiography, etc.), thereafter being employed as a guide with a scope of appropriately selecting a specific route for effecting a therapeutical procedure on an identified pathology, if any.

As shown in FIG. 1, according to one embodiment of the invention, the pathology localizer and therapeutical procedure guide system comprises a generally rectangular elongate strip 1 that is being provided with an arrangement of three linear columnar members 2, 3, 4 having a common point of origin (O) at one end of the strip and extending in the elongate direction till the other end of the same wherein they are correspondingly denoted as axes (X, Y, Z), such linear columnar members 2, 3, 4 constituting a coordination system that is visible in imaging modalities, this visibility being ensured by selection of an appropriate contrast medium that will enhance clarity of three consecutive spots being depicted in sectional images of CT or MRI scanning as imprints of the abovementioned linear columnar members 2, 3, 4 appropriately provided with the contrast medium or of the three linear segments (OX), (OZ), (OY) being depicted in frontal images of X-Ray scanning as imprints of the overall length of the abovementioned linear columnar members 2, 3, 4.

The strip 1 comprises a pair of parallel elongate sides 1a, 1b and a pair of shorter sides 1c, 1d and is produced in various sizes and possibly in shapes other than the preferred rectangular shape to fit particular needs of imaging of different parts of the body.

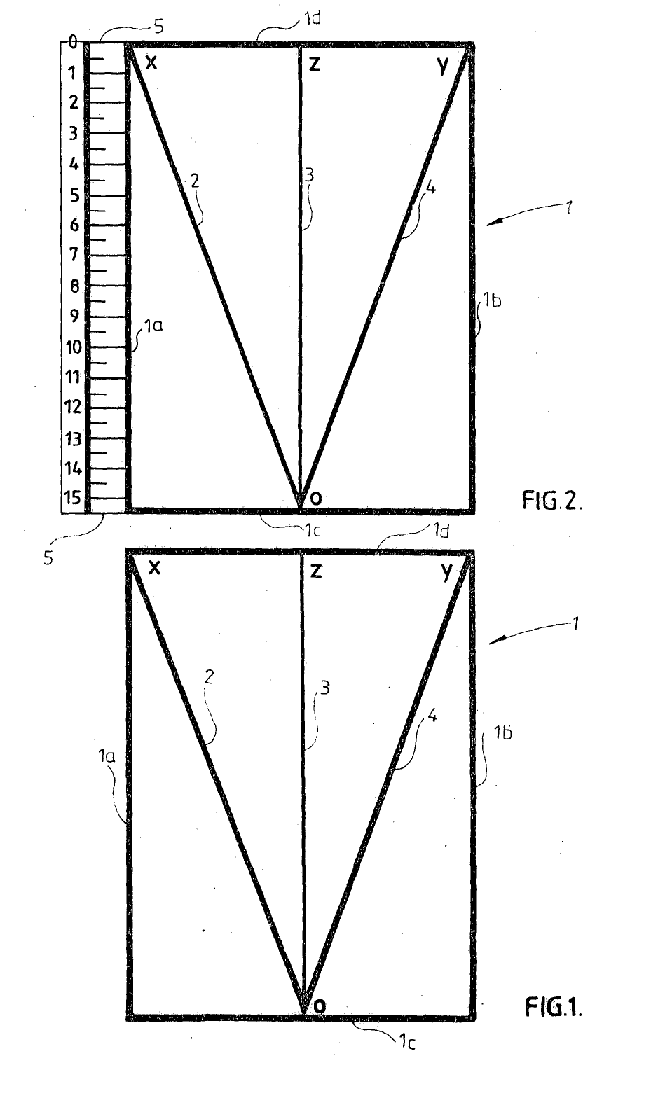

In accordance with a further embodiment of the invention, as shown in FIG. 2, the rectangular elongate strip 1 is also provided with a rule with a scale 5 providing direct recordal of the coordinates of the imprints of the three linear columnar members in the various imaging modalities, wherein this rule with a scale 5 extends along one of the sides of the elongate strip 1, preferably along one of the elongate sides 1a, 1b of the same.

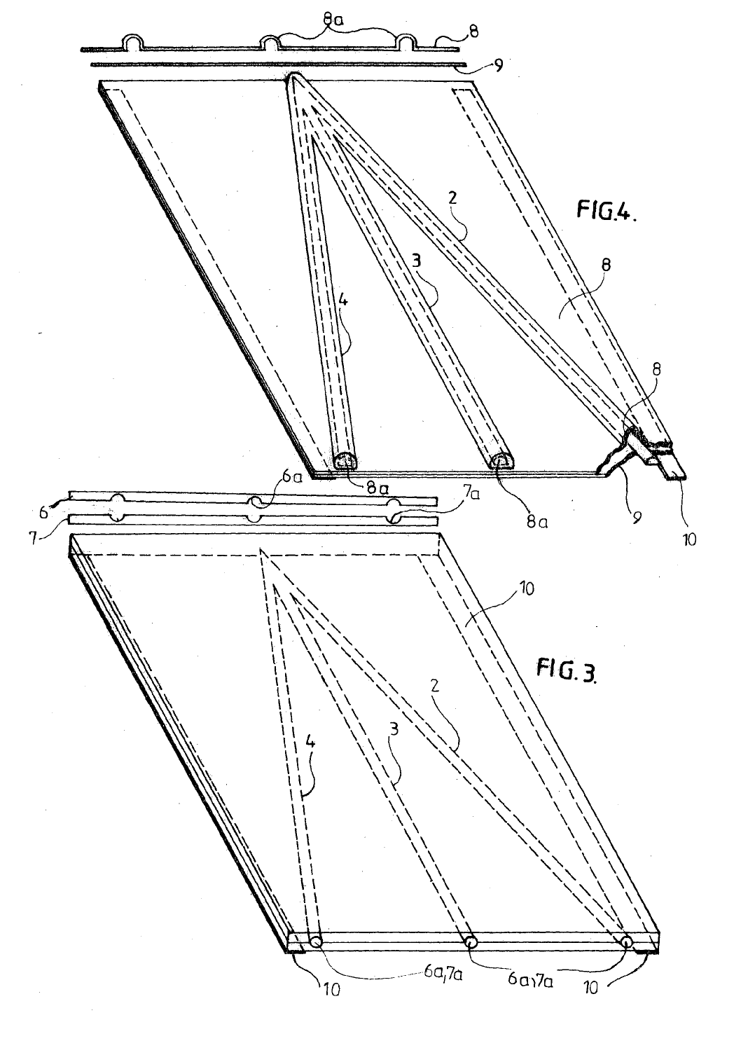

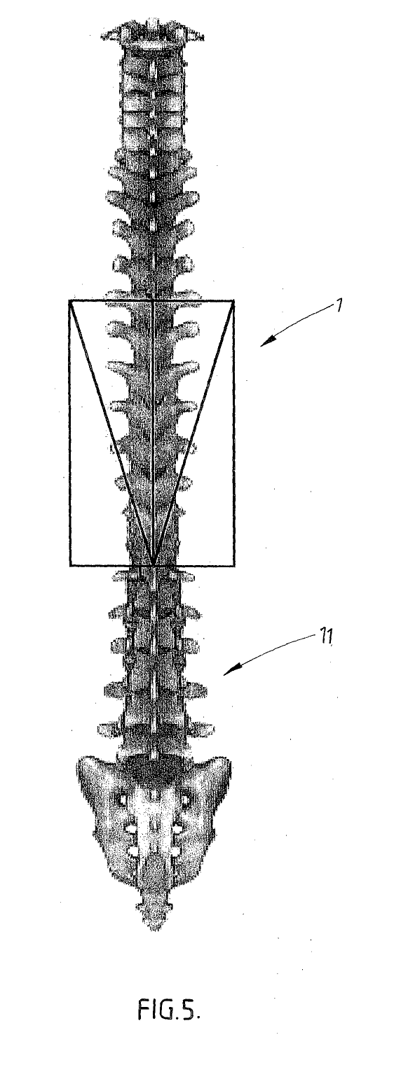

Various materials and various production technologies can be employed in the manufacturing process of the guide strip of the invention. FIGS. 3 and 4 present two illustrative modes of producing the guide strip of the invention.

As shown in FIG. 3 in accordance with an illustrative preferred embodiment of the invention the strip is produced in an extrusion thermosetting process wherein a pair of plastic films 6, 7 are produced, each plastic film 6, 7 comprising semicircular recesses 6a and 7a respectively as shown in the illustrative sectional view of the constituent parts of the strip, such semicircular recesses 6a and 7a extending in the longitudinal direction defined by the elongate sides 1a, 1b of the strip in a convergent mode from side 1d of the strip wherein they are spaced at a maximum distance up to side 1c of the strip wherein they converge all together at the common point of origin (O). When the two plastic films 6, 7 are assembled, the guide strip product of FIG. 3 is produced as the semicircular recesses 6a, 7a match each other and form a circular channel all the way along the linear columnar members 2, 3, 4.

An alternative mode of production of the guide strip of the invention is presented in FIG. 4, wherein a single plastic film 8 undergoes vacuum forming processing in order to incorporate sequential recesses 8a as shown in the illustrative sectional view of the constituent parts of the strip, such recesses 8a also extending in the longitudinal direction defined by the elongate sides la, lb of the strip in a convergent mode from side 1d of the strip wherein they are spaced at a maximum distance up to side 1c of the strip wherein they converge all together at the common point of origin (O). The guide strip product of FIG. 4 is further provided with a non-processed plastic film 9 covering the plastic film 8 that incorporates the sequential recesses 8a.

In both illustrative embodiments of FIG. 3 or FIG. 4 various contrast mediums might be employed to produce the desired outcome of visible marks along and across the linear columnar members 2, 3, 4. By way of example the linear columnar members 2, 3, 4 might be filled with liquid contrast medium, such as gadolinium, broadly used in MRI scanning, wherein when injected into the body, gadolinium contrast medium makes certain tissues, abnormalities or disease processes more clearly visible on a magnetic resonance imaging (MRI) scans. Alternatively a solid contrast medium might be employed, such as a metallic fibre made from paramagnetic material, such as cadmium or tantalum. Furthermore the linear columnar members 2, 3, 4 might also be filled with an appropriate gas contrast medium including ambient air.

According to a preferred embodiment of the invention, the proposed pathology localizer guide strip 1 might be provided with appropriate means adapted to leave a trace onto the body that indicates the exact position in which it was placed prior to the performance of scanning, so that coordinates of the pathology identified might be accurately defined during a posterior surgical or other therapeutical procedure being performed. To this end, the guide strip shown in FIG. 3 or FIG. 4 incorporates bands 10 adapted to dispose an appropriate material onto the skin and thereby leave an adequately durable visible trace therein that will thereafter adequately define the position of placement of the guide strip. At least one band 10 will be employed along at least one of the sides of the guide strip 1. As shown in FIGS. 3 and 4 a pair of bands is illustratively provided along the two elongate sides of the strip. In accordance with a preferred embodiment of the invention these bands 10 might be self-adhesive and also serve the purpose of appropriately sticking the strip onto the body.

The pathology localizer guide strip of the invention reveals a new method of precise localization of pathology and henceforth a new method of accurately guided therapeutical procedure therein.

According to the invention the method of precisely localizing a pathology and of accurately determining an appropriate guide path for the insertion of an invasive or radiotherapy instrument at a targeted site for the performance of a therapeutical procedure thereafter comprises:

A first step in which the doctor examines the patient and determines the appropriate X-Ray or CT or MRI or other scanning required, e.g. in the thoracic or in the lumbar spine with a scope of identifying a suspected pathology.

A second step in which an appropriately sized guide strip is selected and attached along the area that will be subjected to the prescribed X-Ray or CT or MRI or other scanning FIG. 5 illustratively shows a pathology localizer guide strip 1 applied along a specified area of the spine 11 of a human body with a scope of identifying a possible pathology therein.

A third step in which the prescribed X-Ray or CT or MRI or other scanning is being performed and a plurality of images are obtained, whereby an array of visible imprints of three linear segments (OX), (OZ), (OY) corresponding to the three linear columnar members (2, 3, 4) is contained in a frontal image obtained by X-RAY scanning and a series of visible imprints of three sequential spots (X₁, Z₁, Y₁) is contained in each one of a plurality of sectional images obtained by CT or MRI scanning, such three linear segments (OX), (OZ), (OY) or such three sequential spots (X₁, Z₁, Y₁) constituting visual representations of the three linear columnar members (2, 3, 4) being depicted in a frontal X-Ray image or points of intersection of a cross-sectionally directed beam emitted by the CT or MRI scanning apparatus with these three linear columnar members respectively.

If X-ray scanning has been performed, the method further comprises the following steps:

selection of the most appropriate linear segment that intersects the abovementioned three linear segments (OX), (OZ), (OY) and passes through the suspected pathology being identified;

measurement of the pair of consecutive linear segments (XZ) and (ZY) along the abovementioned most appropriate linear segment that intersects the three linear segments (OX), (OZ), (OY) and passes through the suspected pathology being identified;

locating a pair of linear segments (XZ) and (ZY) with equivalent values across the array of the three columnar members 2, 3, 4 extending in the elongate direction of the guide strip 1 that accurately corresponds to the measurement of consecutive linear segments (XZ) and (ZY) along the abovementioned most appropriate linear segment that intersects the three linear segments (OX), (OZ), (OY) and passes through the suspected pathology being identified, thereby establishing a specific linear locus onto the area of the body subjected to imaging, such linear locus accurately defining the coordinates of the identified pathology, and

determining a guide path for the therapeutical instrumentation to be used in a subsequent therapeutical procedure to reach the specified area of the identified pathology, including selection of an entrance point along the specific linear locus defined by the abovementioned most appropriate linear segment that intersects the three linear segments (OX), (OZ), (OY) and passes through the suspected pathology being identified and measuring the angle of penetration parameter determining the guide path for the therapeutical instrumentation to be used in a subsequent therapeutical procedure adapted to reach the targeted point of the identified pathology depicted in the abovementioned frontal X-RAY image.

If CT or MRI scanning has been performed, the method further comprises the following steps:

A fourth step of evaluation of the plurality of sectional images being obtained to select the most appropriate sectional image wherein is located the specific area wherein the suspected pathology is being identified and wherein a subsequent therapeutical procedure will be required.

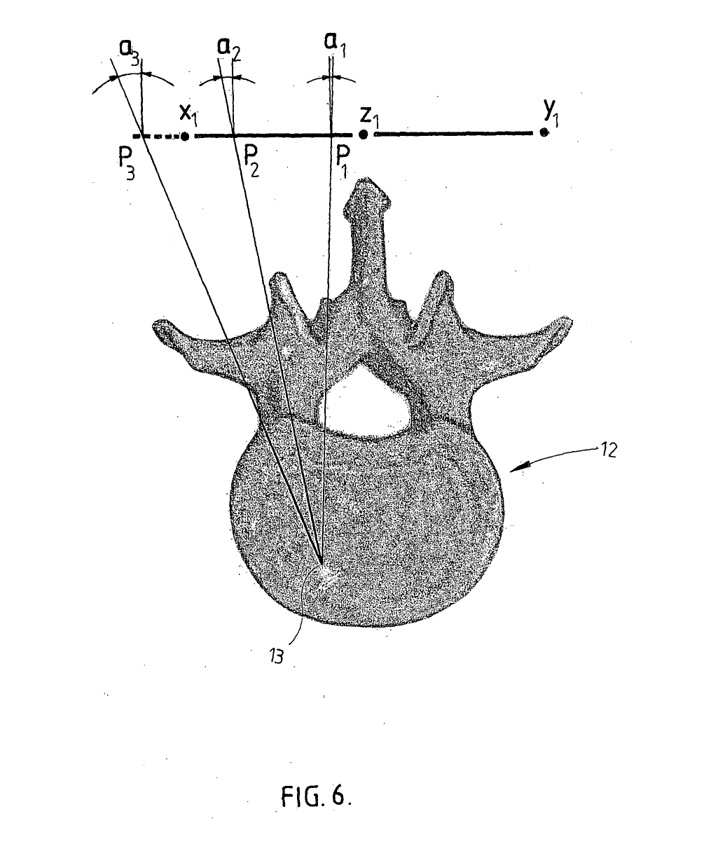

A fifth step of measurement of the pair of consecutive line segments determined by the three sequential spots (X₁, Z₁, Y₁) that constitute the visual representations of the cross-sectionally directed beam emitted by the CT or MRI scanning apparatus intersecting the three linear columnar members 2, 3, 4 extending in the elongate direction of the guide strip 1 in this particularly selected sectional image. FIG. 6 shows a selected sectional image taken by appropriate scanning equipment most appropriately depicting a pathology being identified along the spine, at a location denoted by numeral 13 within a specific vertebra, such sectional image incorporating the three sequential spots (X₁, Z₁, Y₁) that define the specific linear locus of the identified pathology.

A sixth step of locating the pair of linear segments across the array of three columnar members extending in the elongate direction of the guide strip that accurately corresponds to the measurement of the consecutive line segments (X₁Z₁) and (Z₁Y₁) determined by the three sequential spots (X₁, Z₁, Y₁) in the sectional image of FIG. 6 that has been selected hereinabove, thereby establishing a specific linear locus onto the area of the body subjected to imaging, such linear locus accurately defining the coordinates of the identified pathology.

A seventh step during which, if a therapeutical subsequent surgical or radiotherapy procedure is deemed necessary, a guide path for the therapeutical instrumentation being used in the procedure to reach the specified area of the identified pathology at the point denoted by numeral 13 of the vertebra is accurately determined following selection of an entrance point along the hereinabove specific linear locus defined by the three sequential spots (X₁, Z₁, Y₁) and measuring the angle and depth of penetration through measurement of the distance of the selected entrance point from the targeted point of the identified pathology as depicted in the sectional image that has been selected as most appropriately depicting the identified pathology. Thus by way of example FIG. 6 shows three illustrative entrance points (P₁, P₂, P₃), wherein entrance point (P₁) lies almost vertically above the identified pathology 13 and therefore a small angle a₁ of the therapeutical instrumentation is necessary and the depth of insertion thereof is accurately measured by measuring the distance of entrance point (P₁) from the locus of the identified pathology denoted by numeral 13. If entrance point (P₂) is being selected instead then a larger angle a₂ of the therapeutical instrumentation is required and the depth of insertion thereof is in this case also accurately measured by measuring the distance of entrance point (P₂) from the locus of the identified pathology denoted by numeral 13. An entrance point lying beyond the linear segment defined by the sequential spots (X₁, Z₁, Y₁) might also be selected, such as by way of example an entrance point (P₃) and again selection of this entrance point necessitates a yet larger angle a₃ of the therapeutical instrumentation and the depth of insertion thereof is in this case also accurately measured by measuring the distance of entrance point (P₃) from the locus of the identified pathology denoted by numeral 13.

While hereinabove the invention has been described by reference to various preferred embodiments, it is to be appreciated that these are for illustrative purposes only and that those skilled in the art will realize that changes and modifications may be made thereto without departing from the spirit of the invention; it is therefore intended to include such changes and modifications falling within the scope of the invention.

1. Pathology localizer and therapeutical procedure guide system comprising an elongate strip (1) that is being provided with an array of three linear columnar members (2, 3, 4) having a common point of origin (0) at one end of said strip (1) and extending divergently in the elongate direction to corresponding ends (X, Y, Z) at the other end of said elongate strip (1), said three linear columnar members (2, 3, 4) being provided with a contrast medium that is visible in imaging modalities of a body portion with said strip (1) placed thereupon producing an array of visible imprints of three linear segments (OX), (OZ), (OY) corresponding to said three linear columnar members (2, 3, 4) in a frontal image obtained by X-RAY scanning and a series of visible imprints of three sequential spots (X₁, Z₁, Y₁) in a sectional image obtained by CT or MRI scanning, said three linear segments (OX), (OZ), (OY) or said three sequential spots (X₁, Z₁, Y₁) being employed in precisely determining the locus of a pathology, if any, identified within said body portion with said strip (1) placed thereupon and in subsequently determining an accurately defined path of instrumentation adapted to effect a therapeutical procedure therein.

2. Pathology localizer and therapeutical procedure guide system as claimed in claim 1, wherein said elongate strip (1) is generally rectangular and comprises a pair of parallel elongate sides (1a, 1b) and a pair of shorter sides (1c, 1d) with dimensions adapted to fit particular needs of imaging of different parts of the body.

3. Pathology localizer and therapeutical procedure guide system as claimed in claim 2, wherein said strip (1) is manufactured in an extrusion thermosetting process with a pair of plastic films (6, 7), each one of said plastic films (6, 7) comprising semicircular recesses (6a, 7a) respectively, said semicircular recesses (6a, 7a) extending divergently in the longitudinal direction defined by said elongate sides (1a, 1b) of the strip from said common point of origin (O) located along said shorter side (1c) to said corresponding ends (X, Y, Z) located along said shorter side (1d) wherein they are spaced at a maximum distance, whereby as said plastic films (6, 7) are assembled, said semicircular recesses (6a, 7a) match each other and form circular channels all the way along said three linear columnar members (2, 3, 4).

4. Pathology localizer and therapeutical procedure guide system as claimed in claim 2, wherein said strip (1) is manufactured in a vacuum forming process wherein a single plastic film (8) undergoes vacuum forming processing and incorporates sequential recesses (8a), said recesses (8a) extending divergently in the longitudinal direction defined by said elongate sides (1a, 1b) of the strip from said common point of origin (O) located along said shorter side (1c) to said corresponding ends (X, Y, Z) located along said shorter side (1d) wherein they are spaced at a maximum distance, a non-processed plastic film (9) covering said plastic film (8) during assembly of said guide strip product whereby said sequential recesses (8a) define channels all the way along said three linear columnar members (2, 3, 4).

5. Pathology localizer and therapeutical procedure guide system as claimed in claim 1, said elongate strip (1) further comprising a side rule with a scale (5) providing direct recordal of the coordinates of the imprints of said three linear columnar members in the various imaging modalities and at least one self-adhesive band (10) extending along one of the sides of said elongate strip (1) for sticking the same onto the body portion that is being subjected to a specified imaging modality said self-adhesive band (10) including a material adapted to dispose onto the skin a durable visible trace that thereafter defines the position of placement of said strip (1).

6. Pathology localizer and therapeutical procedure guide system as claimed in claim 1, wherein said contrast medium of said three linear columnar members (2, 3, 4) is gadolinium or other appropriate liquid contrast medium.

7. Pathology localizer and therapeutical procedure guide system as claimed in claim 1, wherein said contrast medium of said three linear columnar members (2, 3, 4) is ambient air or other appropriate gas contrast medium.

8. Pathology localizer and therapeutical procedure guide system as claimed in claim 1, wherein said contrast medium of said three linear columnar members (2, 3, 4) consists of fibres made from cadmium or tantalum or other appropriate paramagnetic metallic contrast medium.

9. Method of pathology localization and of accurately guided therapeutical procedure therein comprising:
a first step in which following examination of a patient the doctor determines the appropriate imaging to be carried out in a specific body portion with a scope of identifying a suspected pathology;
a second step in which an appropriately sized elongate guide strip (1) comprising an array of three linear columnar members (2, 3, 4) having a common point of origin (O) at one end of said elongate strip (1) and extending divergently in the elongate direction to corresponding ends (X, Y, Z) at the other end of said elongate strip (1), said three linear columnar members (2, 3, 4) containing a contrast medium, is positioned onto said specific body portion prior to performance of said appropriate imaging;
a third step in which said appropriate imaging is being performed and the images obtained contain visible imprints of said contrast medium containing three linear columnar members (2, 3, 4), said visible imprints thereafter being used in precisely determining the locus of a pathology, if any, identified within said body portion with said elongate strip (1) placed thereupon and in subsequently determining an accurately defined path of instrumentation adapted to effect a therapeutical procedure therein.

10. Method of pathology localization and of accurately guided therapeutical procedure according to claim 9, wherein said appropriate imaging is X-RAY scanning providing a frontal image that contains an array of visible imprints of three linear segments (OX), (OZ), (OY) corresponding to said three linear columnar members (2, 3, 4), said imprints being employed in precisely determining the locus of a pathology, if any, identified within said body portion with said elongate strip (1) placed thereupon and in subsequently determining an accurately defined path of instrumentation adapted to effect a therapeutical procedure therein, further comprising the steps of:
selection of the most appropriate linear segment that intersects said three linear segments (OX), (OZ), (OY) and passes through the suspected pathology being identified;
measurement of the pair of consecutive linear segments (XZ) and (ZY) along said most appropriate linear segment that intersects said three linear segments (OX), (OZ), (OY) and passes through the suspected pathology being identified;
locating the pair of linear segments (XZ) and (ZY) across the array of three columnar members extending in the elongate direction of the guide strip (1) that accurately corresponds to the measurement of said consecutive linear segments (XZ) and (ZY) along said most appropriate linear segment that intersects said three linear segments (OX), (OZ), (OY) and passes through the suspected pathology being identified, thereby establishing a specific linear locus onto the area of the body subjected to imaging, said linear locus accurately defining the coordinates of the identified pathology, and
determining a guide path for the therapeutical instrumentation to be used in a subsequent therapeutical procedure to reach the specified area of the identified pathology, including
selection of an entrance point along said specific linear locus defined by said most appropriate linear segment that intersects said three linear segments (OX), (OZ), (OY) and passes through the suspected pathology being identified;
measuring the angle of penetration parameter determining the guide path for the therapeutical instrumentation to be used in a subsequent therapeutical procedure adapted to reach the targeted point of the identified pathology depicted in said frontal X-RAY image.

11. Method of pathology localization and of accurately guided therapeutical procedure according to claim 9, wherein said appropriate imaging is CT or MRI scanning providing a plurality of sectional images, each sectional image being provided with a series of visible imprints of three sequential spots (X₁, Z₁, Y₁) produced by a cross-sectionally directed beam emitted by the CT or MRI scanning apparatus intersecting said three linear columnar members (2, 3, 4), further comprising the steps of:
evaluation of the plurality of sectional images being obtained to select the most appropriate sectional image wherein is located the specific area wherein the suspected pathology is being identified and wherein a subsequent therapeutical procedure will be required;
measurement of the pair of consecutive linear segments (X₁Z₁) and (Z₁Y₁) determined by the three sequential spots (X₁, Z₁, Y₁) that constitute the visual representations of the cross-sectionally directed beam emitted by the CT or MRI scanning apparatus intersecting said three linear columnar members (2, 3, 4) in this particularly selected sectional image, said three sequential spots (X₁, Z₁, Y₁) determining a specific linear locus of the identified pathology;
locating the pair of linear segments (X₁Z₁) and (Z₁Y₁) across the array of three columnar members extending in the elongate direction of the guide strip that accurately corresponds to the measurement of the consecutive linear segments (X₁Z₁) and (Z₁Y₁) determined by the three sequential spots (X₁, Z₁, Y₁) in said particularly selected sectional image, thereby establishing a specific linear locus onto the area of the body subjected to imaging, said linear locus accurately defining the coordinates of the identified pathology, and
determining a guide path for the therapeutical instrumentation to be used in a subsequent therapeutical procedure to reach the specified area of the identified pathology, including
selection of an entrance point along said specific linear locus defined by said three sequential spots (X₁, Z₁, Y₁);
measuring the angle and depth of penetration parameters determining the guide path for the therapeutical instrumentation to be used in a subsequent therapeutical procedure through measurement of the distance of the selected entrance point from the targeted point of the identified pathology depicted in said particularly selected sectional image.
